(19) United States
(12) Patent Application Publication
(10) Pub. No.: US 2004/0265954 A1
(43) Pub. Date: Dec. 30, 2004

(54) System and method for the production of recombinant proteins

(76) Inventors: Markus Aebi, (US); Michael Wacker, (US)

Correspondence Address: MOETTELI & ASSOCIES SARL, CASE POSTALE 486, AVE DE FRONTENEX 6, GEVEVA 12, CH-1211, CH

(21) Appl. No.: 10/383,673

(22) Filed: Mar. 5, 2003

Related U.S. Application Data
(60) Provisional application No. 60/364,655, filed on Mar. 14, 2002.

(30) Foreign Application Priority Data
Mar. 7, 2002 (CH) 2002 0394/02

(51) Int. Cl. C12P 21/02; C12N 1/21; C07K 14/195; C07K 14/47; C12N 9/10

(52) U.S. Cl. 435/69.1; 435/488; 435/252.33; 435/320.1; 530/395; 536/23.2; 435/193

(57) ABSTRACT

A system and a method for the production of recombinant N-glycosylated target proteins. The system comprises a prokaryotic organism (e.g. Escherichia coli) into which is introduced a genetic information encoding for a metabolic apparatus capable of carrying out the requested N-glycosylation of the target protein. Said prokaryotic organism also contains the genetic information required for the expression of one or more recombinant target proteins. The metabolic apparatus preferably comprises specific glycosyltransferases for the assembly of the oligosaccharide on a lipid carrier and an OTase that covalently links this oligosaccharide to specific residues of the desired protein.

### RELATED PATENT APPLICATIONS

This application claims priority of the Swiss patent application No. 0394/02 filed Mar. 7, 2002 and of the U.S. provisional application No. 60/364,655 filed Mar. 14, 2002.

### FIELD OF INVENTION

The present invention relates to an expression system and a method for the production of recombinant human and/or animal and/or plant and/or prokaryotic and/or fungal glycoproteins. Such glycoproteins may serve as nutrition or medical drugs for human or animals or plants because of their identical structure to the glycoproteins normally produced in these organisms.

### TECHNICAL BACKGROUND

Glycosylation constitutes one of the most important of all post-translational protein modifications in eukaryotic cells and may have numerous effects on function, structure, physical properties and targeting of particular proteins. Generally, the carbohydrate moiety is to be regarded as having significant effects on both the structure and on the physicochemical features of a protein and may affect its enzymatic activity, antigenicity or thermal stability. The sugars can be linked via the Famine group of an asparagine (N-glycosidic bond) or the hydroxyl group of a serine or threonine (O-glycosidic bond) residue.

The N-linked protein glycosylation is by far the most common protein modification found in eukaryotes. The complex glycosylation process starts at the cytoplasmic face of the endoplasmatic reticulum (ER) with the assembly of an oligosaccharide on the lipid carrier dolichylpyrophosphate [Burda, P. and Aebi, M. (1999) The dolichol pathway of N-linked glycosylation. Biochim Biophys Acta, 1426, 239-257]: 2 N-acetylglucosamine and 5 mannose residues are attached to this lipid in a stepwise fashion. The lipid linked oligosaccharide (LLO) is then flipped into the lumen of the ER, where by addition of 4 mannose and 3 glucose residues full length LLO is obtained. In the central reaction of the process, this oligosaccharide is transferred to selected asparagine residues of newly synthesized polypeptides. This reaction is catalyzed by the oligosaccharyl transferase (OTase) in the lumen of the ER. The OTase is a complex of at least 8 subunits and this enzyme is responsible for the formation of the N-glycosidic bond. While still in the ER, three glucose and one mannose residue are quickly removed from the oligosaccharide of the glycoprotein. Glycoproteins are then transported to the Golgi apparatus where further trimming and addition of sugar moieties occurs before they are targeted to their final destinations [Varki, A., Cummings, R., Esko, ]., Freeze, H., Hart, G. and Marth, J. (1999) Essentials of Glycobiology. Cold Spring Harbor Laboratory Press, Cold Spring Harbor, N.Y.]. Whereas the LLO synthesis is a highly conserved process in eukaryotic cells, the modifications in the Golgi are not only species specific but also cell-type specific and lead to a high degree of diversity with respect to the structure of the N-linked oligosaccharides.

### PRIOR ART

The production of human proteins in a variety of heterologous expression systems has become an important technique to generate recombinant proteins for research purposes as well as pharmaceutical applications. It is generally recognized that there is no universal expression system available for production. Furthermore, the selection of a cell type for expression of heterologous proteins depends on many factors. These include criteria such as cell growth characteristics, expression levels, intracellular or extracellular expression, and biological activity of the protein of interest as well as its intended use. But one of the most important criteria to be considered is whether a protein needs to be glycosylated for its application. Many human therapeutics are glycoproteins, and the importance of the posttranslational modification of polypeptides with defined oligosaccharides is well documented by their implication in numerous biological phenomena.

In mammalian glycoproteins, the majority of N-glycans are of the complex type, i.e. they consist of a pentasaccharide core of two N-acetylglucosamine and three mannose residues. This core is the remaining structure of the oligosaccharide that was originally transferred from dolichylpyrophosphate to proteins. In the Golgi it is further modified with antennae comprising additional N-acetylglucosamine, galactose, sialic acid and often fucose residues. An enormous diversity of impressively complex oligosaccharide structures is thereby possible. The importance of the complex N-glycans and the essential role of these structures are shown with experiments using knock-out mice unable to synthesize these complex-type N-glycans. These mice die before birth. Recently the congenital disorder of glycosylation (CDG) has been described in humans. This is a group of congenital multi-systemic diseases characterized by deficiency in the generation of N-glycans. These disorders are manifested by a wide variety of clinical features, such as disorders of the nervous system development, psychomotor retardation, dysmorphic features, hypotonia, coagulation disorders, and immunodeficiency. The broad spectrum of features reflects the critical role of N-glycoproteins during embryonic development, differentiation, and maintenance of cell functions and emphasizes the importance of the correct oligosaccharide structure.

Since the majority of therapeutically relevant proteins are glycosylated in their natural forms, they should also be glycosylated as recombinant proteins in order to get the correct biological activity. Thus, monitoring of the glycosylation pattern in quality control of recombinant proteins to assure product safety, efficiency and consistency has become increasingly important. Systems for the expression of glycosylated proteins have been developed. The most commonly used are Chinese hamster ovary (CHO) cell lines [Grabenhorst, E., Schlenke, P., Pohl, S., Nimtz, M. and Conradt, H. S. (1999) Genetic engineering of recombinant glycoproteins and the glycosylation pathway in mammalian host cells. Glycoconjugate Journal, 16, 81-97], insect cells [Altmann, F., Staudacher, E., Wilson, I. B. and Marz, L. (1999) Insect cells as hosts for the expression of recombinant glycoproteins. Glycoconjugate Journal, 16, 109-123] or fungal cells [Malissard, M., Zeng, S. and Berger, E. G. (1999) The yeast expression system for recombinant glycosyltransferases. Glycoconjugate Journal, 16, 125-139. or Maras, M., van Die, I., Contreras, R. and van den Hondel, C. A. (1999) Filamentous fungi as production organisms for glycoproteins of bio-medical interest. Glycoconjugate Journal, 16, 99-107]. These cell lines have all the capability to glycosylate proteins, but they exhibit major differences in the production of recombinant glycoproteins. As mentioned above, the synthesis of the LLO and the transfer of the oligosaccharide to polypeptides is a highly conserved mechanism in all eukaryotes, whereas further processing and trimming of the N-glycans in the Golgi vary between organisms and cell type. Therefore, the final structure of the recombinant glycoprotein is defined by the production cell used. CHO cells are the mammalian cell lines commonly used for the expression of recombinant glycoproteins. They are able to synthesize complex type N-glycans, but some human tissue-specific terminal sugar residues are not synthesized by these cells since they do not express the proper glycosyltransferases. Therefore, the host cell lines must be improved by genetic engineering with the introduction of these glycosyltransferases. Insect cells [see also WO 00/52135] are also widely used to produce recombinant proteins, as they can synthesize large quantities of a protein of interest when infected with powerful baculovirus-based gene expression vectors, and they can provide post-translational modifications similar to those provided by mammalian cells. The N-glycosylation pathway parallels the mammalian pathway until the formation of the core pentasaccharide. But normally insect cells do not express additional transferases in the-Golgi and therefore the N-glycans produced are truncated (paucimannosidic) instead of a complex type as found in mammalian cells.

Fungi, in particular Saccharomyces cerevisiae or Pichia pastoris, are suitable host organisms for the production of eukaryotic heterologous proteins. These systems combine well-known techniques for the molecular and genetic manipulations, the cells are easy to grow and they have the capability for complex post-translational modifications such as protein glycosylation. In contrast to animal cells, fungi do not further trim the oligosaccharide in the Golgi but instead elongate it directly by the addition of mannose residues to form mannanes with up to 200 mannose units. Some glycoproteins escape these modifications and their maturation is more limited, yielding short core type oligosaccharides with up to 13 mannose residues.

These three examples of N-glycosylation in eukaryotes emphasize the differences in the structure of N-glycans. The implication on the function reveals that exact analysis of the structure is essential. Significant advances in carbohydrate structural analyses have been achieved during the past years. Especially in mass spectrometry (on-line ESI-MS, nanospray tandem mass spectrometry (ESI-MS/MS) and improved MALDI/TOF techniques), very sensitive instrumentation for glycosylation analysis has been made available.

### PROBLEMS IN PRIOR ART

The importance of a highly defined oligosaccharide structure on recombinant glycoproteins contrasts sharply to the inability of presently used biotechnological processes to generate glycoproteins. This is due to the fact that the structure of a protein-linked oligosaccharide is determined directly by the cell type used and all of the eukaryotic production systems exhibit this specificity. It might be possible to genetically engineer eukaryotic production cell lines in such a way that a defined oligosaccharide structure is produced. However, the plethora of glycosyltransferases active in the Golgi compartment of eukaryotes makes such an approach very difficult. Additional problems with the use of eukaryotic expression systems are the following: In general, the mammalian expression system has its drawbacks in the use of growth medium, which contains calf serum. This raises concern about biosafety because of possible contamination with bovine spongiform encephalopathy (BSE). Furthermore human cell line cultures are much more difficult to keep sterile, these cells grow slowly and require expensive process control. As mentioned before, the specific glycoprotein synthesized depends directly on the cell line or cell type used. In other words, a recombinant glycoprotein only gets modified with its original N-glycan if the heterologous system expresses the same enzymes of the N-glycosylation pathway as in its origin. Otherwise host cells must be adapted by genetic engineering of the glycosylation pathway in the Golgi (FIG. 1B), and this represents the major drawback of human cell lines in the expression of recombinant glycoproteins.

The difficulties of the production of recombinant proteins in insect cells with the help of the baculoviruses expression system are the following: Baculoviruses essentially have a lytic infection mode, i.e. when the product is harvested, a large proportion of the host cells is lysed and releases degradative enzymes. In addition, the protein synthesis is maximal near death of infected cells and it is possible that the overall processing of the protein is suboptimal at that time. Particularly proteins destined for the plasma membrane or for secretion are affected by the depletion of components of the post-translational machinery of the secretory pathway. Furthermore, large scale insect cell culture offers particular challenges to the biotechnologist due to the higher oxygen consumption and higher shear sensitivity of the cells as compared to mammalian cells. Like in mammalian cells, the major drawback in the heterologous expression of glycoproteins resides in the different structure of the N-glycan as described before. Especially the lack of terminal sialic acid residues is detrimental, because these sugars play important roles in glycoprotein biology.

To summarize, the three main eukaryotic expression systems mostly fail to produce glycans of a desired structure. In contrast to eukaryotic systems, the gram-negative bacterium Escherichia coil offers several technical advantages for the production of heterologous proteins. It is the oldest and most productive system used. However, the inability of E. coli cells to exert post-translational modifications of proteins remains the strongest drawback for its use as the preferred host for the production of human proteins.

### SUMMARY OF THE INVENTION

To overcome the problem of production of recombinant glycoproteins in E. coli, a metabolic machinery capable to obtain protein glycosylation is introduced into this bacterium. It is therefore an object of the invention to provide an expression system with which recombinant proteins, in particular N-glycosylated proteins are producible.

This object of the invention is reached—according to a first aspect—by the combination of features of independent claim 1, wherein a system is proposed for the production of recombinant human, human-like, or animal, or plant, or fungal, or bacterial N-glycosylated target proteins, the system comprising a prokaryotic organism into which is introduced a genetic information encoding for a metabolic apparatus capable of carrying out the requested N-glycosylation of the target protein. The system according to the invention is characterized in that said prokaryotic organism also contains the genetic information required for the expression of one or more recombinant target proteins.

This object of the invention is reached—according to a second aspect—by the combination of features of independent claim 5, wherein a method is proposed for producing recombinant human, human-like, or animal, or plant, or fungal, or bacterial N-glycosylated target proteins, the system comprising a prokaryotic organism into which is introduced a genetic information encoding for a metabolic apparatus capable of carrying out the requested N-glycosylation of the target protein. The method according to the invention is characterized in that said prokaryotic organism also contains the genetic information required for the expression of one or more recombinant target proteins.

Additional and inventive features derive from the dependent claims.

### ADVANTAGES OVER PRIOR ART

Since E. coli is easier to handle and to grow and its genetics are very well known, the production of human, human-like, animal or plant or fungal or bacterial glycoproteins in E. coli is a breakthrough in biotechnology.

As mentioned before, recombinant glycoproteins to date have to be produced in less suited eukaryotes. But although the first steps in the synthesis of N-glycoproteins are highly conserved in all organisms the further trimming and processing differs quite significantly between eukaryotes. Therefore the N-glycans of recombinant glycoproteins depend on the glycosylation genes present in the expression system used.

This could give rise to production of recombinant glycoproteins where the N-glycans differ in their structure compared to the original one.

In contrast, the introduction of a genetic information encoding for a metabolic apparatus capable of carrying out the requested glycosylation of the protein, e.g. an operon, into an organism that normally does not glycosylate proteins offers the opportunity to manipulate the structure of the N-glycan by introducing specific glycosyltransferases.

### BRIEF DESCRIPTION OF THE DRAWINGS

Some of the problems known from prior art as well as the method according to the invention are explained in more detail referring to schematic drawings that are exemplary embodiments of the invention and are not intended to narrow the scope of protection of the present invention. There is shown in

FIG. 1 the expression of recombinant glycoproteins in eukaryotes, whereas FIG. 1A shows the expression of a target glycoprotein, and FIG. 1B shows genetic engineering of existing glycosylation pathways in the Golgi;

FIG. 2 the Escherichia coli expression system, with the expression of a recombinant target protein and the introduction of a specific glycosylation pathway according to the invention; and in

FIG. 3 the legend for the signs representing individual elements of the oligosaccharides residues of the glycoproteins in FIG. 1 and FIG. 2.

### DESCRIPTION OF THE INVENTION

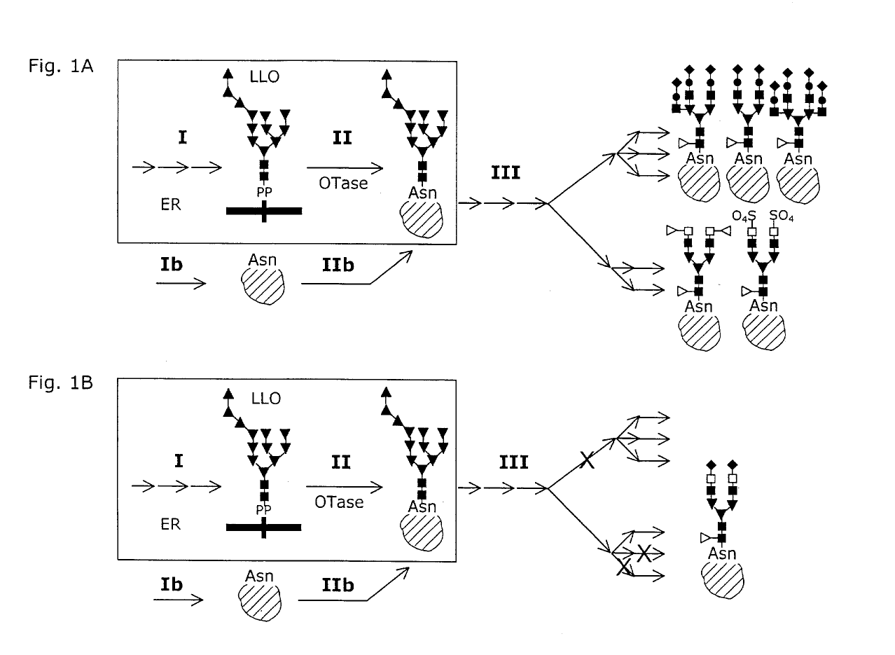

FIG. 1 shows the expression of recombinant glycoproteins in eukaryotic expression systems. FIG. 1A shows the expression of a target glycoprotein, wherein the assembly of the lipid linked oligosaccharide (LLO; step I) and the transfer of the oligosaccharide to the protein by means of an OTase (step II) is a highly conserved process in the Endoplasmatic Reticulum (ER). In contrast, the modifications in the Golgi are cell type specific (step III).

FIG. 1B again shows the expression of a target glycoprotein, wherein the assembly of the lipid linked oligosaccharide (LLO; step I) and the transfer of the oligosaccharide to the protein by means of a OTase (step II) is a highly conserved process in the ER. In addition, an attempt to carry out genetic engineering of existing glycosylation pathways in the Golgi is shown. To produce a recombinant protein with a specific structure in eukaryotic cells, the host cells have to be adapted by genetic engineering of this glycosylation pathway in the Golgi. “X”-signs mark deletions required to exclude undesired pathways. “Asn” indicates an asparagine, “PP” a pyrophosphate, and “SO₄” a sulfate group.

Both FIGS. 1A and 1B show that the expression of the recombinant protein is carried out outside the ER (step Ib) and that this target protein then is imported into the ER (step IIb). The explanation of the signs representing the individual elements of the oligosaccharides derives from the legend in FIG. 3.

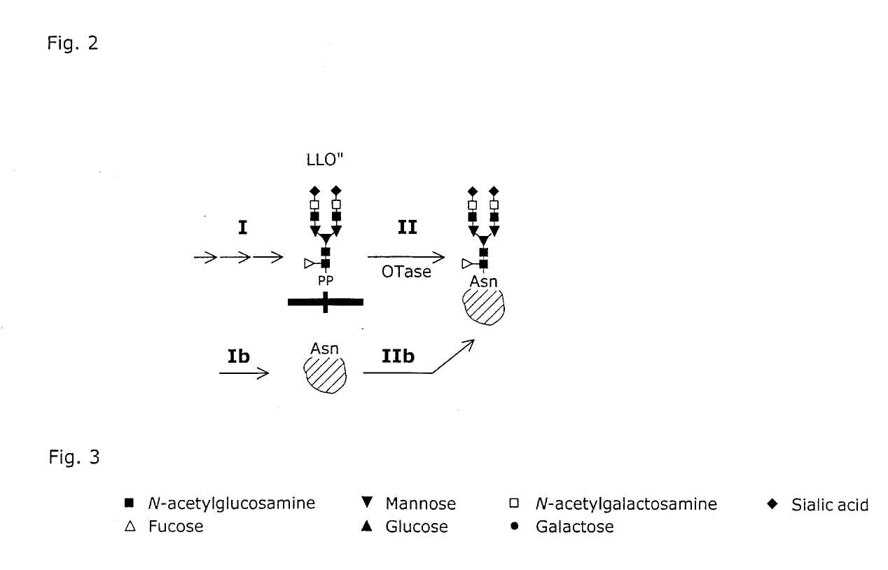

To obtain a recombinant glycoprotein with a specific oligosaccharide structure in eukaryotic cells requires the tailoring of highly complex, essential pathways and this might possibly interfere with the viability of the production cell. This is not the case in the E. coli system. Here, the tailoring is obtained by the introduction of specific components of the glycosylation machinery that lead to the desired glycoprotein (FIG. 2).

Since all the basic components (monosaccharides) required for the assembly of oligosaccharides are present in E. coli cells, the above mentioned solution requires the introduction:

a) of specific glycosyltransferases for the assembly of the oligosaccharide on a lipid carrier, and

b) an OTase that covalently links this oligosaccharide to specific residues of the desired protein.

This solution offers the possibility to design the oligosaccharide structure by the expression of specific glycosyltranferases and does not affect vital functions of the production cell.

FIG. 2 shows the Escherichia coli expression system according to the invention with the expression of a recombinant target protein (step Ib), which then is introduced to the glycoprotein synthesis (step IIb). To obtain a specific glycoprotein in E. coli, specific glycosyltransferases for the assembly of the lipid-linked oligosaccharide (LLO″; step I) are introduced into the host. The OTase covalently links this oligosaccharide to specific residues of the desired protein (step II).

In another attempt, the oligosaccharide, that is attached to the desired protein as described in FIG. 2, can be exchanged using a different oligosaccharide as a substrate in a enzymatic reaction-in vitro. It was shown that the immobilized endo-p-N-acetylglucosaminidase (Endo-A) from Arthrobacter protophormiae could transfer an oligosaccharide to ribonuclease B that contained a covalently linked N-acetylglucosamine [Fujita, K., Tanaka, N., Sano, M., Kato, I., Asada, Y. and Takegawa, K. (2000) Synthesis of neoglycoenzymes with homgogenous N-linked oligosaccharides using immobilized endo-β-N-acetylglucosaminidase A. Biochemical and Biophysical Research Communications, 267, 134-13]. Thus the invention gives the possibility to produce a glycoprotein in E. coli and then, in a second step, to modify the oligosaccharide that is covalently linked to the protein by exchanging it with a different oligosaccharide of defined structure with the immobilized Endo-A in vitro.

The invention encompasses the production of glycosylated glycoproteins. There are many benefits derived from the glycosylation of such proteins. Such benefits include, but are not limited to, increased in vivo circulatory half life of a protein; increased yields of recombinant proteins; increased biological activity of the protein including, but not limited to, enzyme activity, receptor activity, binding capacity; altered antigenicity; improved therapeutic properties; increased capacities as a vaccine or a diagnostic tool, and the like. Examples of mammalian glycoproteins that can be produced with this invention and that can serve as medicaments for humans, animals or plants, include but are not limited to, erythropoietin, transferrin, interferons, immunoglobulines, interleukins, plasminogen, and thyrotropin. Also prokaryotic and/or fungal glycoproteins can be produced with the invention and can serve as medicaments for humans, animals and plants, e.g. glycoproteins from C. jejuni and from fungi. Further applications for glycoproteins produced with this invention include, but are not limited to, industrial enzymes, functional food, cosmetics, packaging materials, and textiles.

### EXAMPLE 1

The present invention bases on the finding, that Campylobacter jejuni, a gram-negative bacterium, produces glycoproteins. Utilizing methods known per se, we have introduced the C. jejuni gene encoding AcrA, a glycoprotein, into E. coli. This results in the expression of non-glycosylated AcrA protein (see FIG. 2, step Ib). Subsequently and again utilizing known methods, an operon of C. jejuni encoding a) specific glycosyltransferases and b) an OTase was introduced into E. coli. This resulted in the production of specifically glycosylated AcrA protein according to the invention (see FIG. 2, steps I and II), as verified—always using methods known to skilled persons—by the binding of a highly specific lectin and glycosylation specific antibodies to the heterologously produced AcrA protein [Michael Wacker et al. (2002) N-linked glycosylation in Campylobacter jejuni and its functional transfer into E. coli (SCIENCE, Vol 298: 1790-1793]. In addition, the structure of the oligosaccharide linked to AcrA was verified by mass spectroscopy. Next it was shown, that the oligosaccharide was only transferred to the ε-amino group of the asparagine within the consensus sequence Asn-X-Ser/Thr where X can be any amino acid except Pro [Gavel, Y. and Von Heijne, G. (1990). Sequence differences between glycosylated and non-glycosylated Asn-X-Thr/Ser acceptor sites: implications for protein engineering. Protein Eng, 3, 433-442]. When the consensus sequence was mutated, the oligosaccharide was not transferred to the protein anymore. Therefore, it was verified—always using methods known to skilled persons—that the OTase of C. jejuni recognized the same consensus sequence as the OTase of eukaryotes and archaea and transferred the oligosaccharide by the same proposed mechanism to the protein [Wacker, M., Linton, D., Hitchen, P. G., Nita-Lazar, M., Haslam, S. M., North, S. J., Panico, M., Morris, H. R., Dell, A., Wren, B. W. and Aebi, M. (2002). N-linked glycosylation in Campylobacter jejuni and its functional transfer into E. coli. Science, 298: 1790-1793].

Specific glycosyl transferases and oligosaccharyl transferases utilized to genetically modify E. coli can be of prokaryotic or eukaryotic origin as glycosyl transferases are ubiquiteous and oligosaccharyl transferases are known from archaea.

What is claimed is:

1. A system for the production of recombinant N-glycosylated target proteins, the system comprising a prokaryotic organism into which is introduced a genetic information encoding for a metabolic apparatus capable of carrying out the requested N-glycosylation of the target protein, wherein said prokaryotic organism also contains the genetic information required for the expression of one or more recombinant target proteins.

2. The system of claim 1, wherein the metabolic apparatus comprises specific glycosyltransferases for the assembly of the oligosaccharide on a lipid carrier and an OTase that covalently links this oligosaccharide to specific residues of the desired target protein.

3. The system of claim 1, wherein the prokaryotic organism is Escherichia coli.

4. The system of claim 2, wherein the prokaryotic organism is producing N-glycanes with a specific structure which is defined by the type of the specific glycosyltransferases.

5. A method of producing recombinant N-glycosylated target proteins, the method comprising the introduction of a genetic information encoding for a metabolic apparatus capable of carrying out the requested N-glycosylation of the target protein into a prokaryotic organism, wherein also the genetic information required for the expression of one or more recombinant target proteins is introduced into said prokaryotic organism.

6. The method of claim 5, wherein the metabolic apparatus comprises specific glycosyltransferases for the assembly of the oligosaccharide on a lipid carrier and an OTase, the OTase covalently linking this oligosaccharide to specific residues of the desired target protein.

7. The method of claim 5, wherein the prokaryotic organism is Escherichia coli.

8. The method of claim 5, wherein—by selection of specific glycosyltranferases—the prokaryotic organism is producing N-glycanes with a specific structure which is defined by the type of the specific glycosyltransferases.

9. Utilization of the system of one of claims 1-4 or the method of one of claims 5-8 for the production of target proteins for the development of medicaments or for the production of medicaments for the treatment of humans or animals or plant.

10. Proteins for nutrition and/or pharmaceutical purposes, produced with the system of one of claims 1-4 or according to the method of one of claims 5 to 8.

11. Vaccines, Cytokines and the like medicaments for human or animals or plant, produced with the system of one of claims 1-4 or according to the method of one of claims 5 to 8.

12. Industrial enzymes, functional food, cosmetics, packaging materials or textiles comprising proteins produced with the system of one of claims 1-4 or according to the method of one of claims 5-8.

13. Utilization of a medicament produced with the system of one of claims 1-4 or according to the method of one of claims 5-8 for the therapy of human or animal or plant diseases.
